(19) United States
(12) Patent Application Publication
(10) Pub. No.: US 2023/0233800 A1
(43) Pub. Date: Jul. 27, 2023

(54) Reinforced Catheter Tips, Catheters, and Methods Thereof

(71) Applicant: Bard Access Systems, Inc., Salt Lake City, UT (US)

(72) Inventors: Glade H. Howell, Draper, UT (US); Juan Sepulveda, Centerville, UT (US); Daniel B. Blanchard, Bountiful, UT (US)

(21) Appl. No.: 18/098,052

(22) Filed: Jan. 17, 2023

Related U.S. Application Data
(60) Provisional application No. 63/302,838, filed on Jan. 25, 2022.

(51) Int. Cl. A61M 25/00; A61M 25/09

(52) U.S. Cl. CPC A61M 25/0068; A61M 25/09; A61M 25/0097

(57) ABSTRACT

Reinforced catheter tips, catheters including rapidly insertable central catheters (“RICCs”), and methods thereof reduce the number of steps and medical devices involved in introducing catheters into patients. In an example of a catheter, a RICC can include a catheter tube, a catheter hub coupled to a proximal portion of the catheter tube, and a plurality of extension legs extending from the catheter hub. The catheter tube can include a first section in a distal portion of the catheter tube and a second section proximal of the first section. The first section of the catheter tube can distally terminate with a catheter tip reinforced with a reinforcement band therein. The plurality of extension legs that extend from the catheter hub can be equal in number to a plurality of lumens extending through the RICC. In another example, a method can include a method of making the foregoing RICC.

### PRIORITY

This application claims the benefit of priority to U.S. Provisional Application No. 63/302,838, filed Jan. 25, 2022, which is incorporated by reference in its entirety into this application.

### BACKGROUND

Central venous catheters (“CVCs”) are commonly introduced into patients and advanced through their vasculatures by way of the Seldinger technique. The Seldinger technique utilizes a number of steps and medical devices (e.g., a needle, a scalpel, a guidewire, an introducer sheath, a dilator, a CVC, etc.). While the Seldinger technique is effective, the number of steps are time consuming, handling the number of medical devices is awkward, and both of the foregoing can lead to patient trauma. In addition, there is a relatively high potential for touch contamination due to the number of medical devices that need to be interchanged during the Seldinger technique. As such, there is a need to reduce the number of steps and medical devices involved in introducing a catheter such as a CVC into a patient and advancing the catheter through a vasculature thereof.

Disclosed herein are reinforced catheter tips, catheters including rapidly insertable central catheters (“RICCs”), and methods thereof that reduce the number of steps and medical devices involved in introducing catheters into patients.

### SUMMARY

Disclosed herein is a RICC including, in some embodiments, a catheter tube, a catheter hub coupled to a proximal portion of the catheter tube, and a plurality of extension legs extending from the catheter hub. The catheter tube includes a first section in a distal portion of the catheter tube and a second section proximal of the first section. The first section of the catheter tube distally terminates with a catheter tip reinforced with a reinforcement band therein. The plurality of extension legs that extend from the catheter hub are equal in number to a plurality of lumens extending through the RICC.

In some embodiments, the reinforcement band is sufficiently rigid over its length to support aspiration through the RICC without collapsing the catheter tip.

In some embodiments, the reinforcement band is sufficiently rigid over its length to prevent buckling of the catheter tip when the RICC is inserted into a needle tract to a blood-vessel lumen, optionally, over an access guidewire.

In some embodiments, the reinforcement band is formed of a thermoplastic polymer or a metal over which the first section of the catheter tube is molded or extruded.

In some embodiments, the reinforcement band is formed of the thermoplastic polymer. The thermoplastic polymer is selected from a thermoplastic polyurethane, a carbonate-based thermoplastic polyurethane, a polyamide, and a polyamide-block-polyether copolymer.

In some embodiments, the reinforcement band is formed of the metal. The metal is selected from stainless steel, titanium, and nitinol.

In some embodiments, the RICC is a triluminal catheter including a trifurcated catheter hub as the catheter hub and three extension legs for the plurality of extension legs. Each extension leg of the three extension legs includes a Luer connector coupled to a proximal portion of the extension leg.

In some embodiments, the RICC includes a primary lumen, a secondary lumen, and a tertiary lumen. The primary lumen extends from an opening in a proximal end of a first Luer connector to an opening in the catheter tip. The secondary lumen extends from an opening in a proximal end of a second Luer connector to a secondary-lumen opening in a distal portion of the second section of the catheter tube. The tertiary lumen extends from an opening in a proximal end of a third Luer connector to a tertiary-lumen opening in the distal portion of the second section of the catheter tube proximal of the secondary-lumen opening.

In some embodiments, each lumen of the secondary lumen and the tertiary lumen terminates at a distal end of the second section of the catheter tube.

In some embodiments, a proximal end of the first section of the catheter tube is bonded to the distal end of the second section of the catheter tube.

In some embodiments, the first section of the catheter tube is formed of a first polymeric material having a first durometer and the second section of the catheter tube is formed of a second polymeric material having a second durometer less than the first durometer.

In some embodiments, each polymeric material of the first and second polymeric materials is a polyurethane.

In some embodiments, each lumen of the secondary lumen and the tertiary lumen terminates within a medial portion of the first section of the catheter tube.

In some embodiments, an outer layer of the catheter tube is extruded over an inner layer of the catheter tube. The inner layer of the catheter tube includes the reinforcement band thereover.

In some embodiments, the first section of the catheter tube is tapered from the second section of the catheter tube to the catheter tip. Differently, the second section of the catheter tube has a constant outer diameter.

Also disclosed herein is a catheter tube including, in some embodiments, a first section in a distal portion of the catheter tube and a second section proximal of the first section. The first section of the catheter tube has a single catheter-tube lumen passing therethrough. In addition, the first section of the catheter tube distally terminates with a catheter tip reinforced with a reinforcement band therein. The second section of the catheter tube has two or more catheter-tube lumens passing therethrough including a same catheter-tube lumen as the catheter-tube lumen passing through the first section of the catheter tube.

In some embodiments, the reinforcement band is sufficiently rigid over its length to support aspiration through the catheter tube without collapsing the catheter tip.

In some embodiments, the reinforcement band is sufficiently rigid over its length to prevent buckling of the catheter tip when the catheter tube is inserted into a needle tract to a blood-vessel lumen, optionally, over an access guidewire.

In some embodiments, the reinforcement band is formed of a thermoplastic polymer or a metal over which the first section of the catheter tube is molded or extruded.

In some embodiments, the reinforcement band is formed of the thermoplastic polymer. The thermoplastic polymer is selected from a thermoplastic polyurethane, a carbonate-based thermoplastic polyurethane, a polyamide, and a polyamide-block-polyether copolymer.

In some embodiments, the reinforcement band is formed of the metal. The metal is selected from stainless steel, titanium, and nitinol.

In some embodiments, the catheter tube is a triluminal catheter tube including a primary catheter-tube lumen, a secondary catheter-tube lumen, and a tertiary catheter-tube lumen. The primary catheter-tube lumen corresponds to the catheter-tube lumen passing through both the first and second sections of the catheter tube. The primary catheter-tube lumen extends from an opening in a proximal end of the second section of the catheter tube to an opening in the catheter tip. The secondary catheter-tube lumen extends from an opening in the proximal end of the second section of the catheter tube to a secondary-lumen opening in a distal portion of the second section of the catheter tube. The tertiary catheter-tube lumen extends from an opening in the proximal end of the second section of the catheter tube to a tertiary-lumen opening in the distal portion of the second section of the catheter tube proximal of the secondary-lumen opening.

In some embodiments, each catheter-tube lumen of the secondary catheter-tube lumen and the tertiary catheter-tube lumen terminates at a distal end of the second section of the catheter tube.

In some embodiments, a proximal end of the first section of the catheter tube is bonded to the distal end of the second section of the catheter tube.

In some embodiments, the first section of the catheter tube is formed of a first polymeric material having a first durometer and the second section of the catheter tube is formed of a second polymeric material having a second durometer less than the first durometer.

In some embodiments, each polymeric material of the first and second polymeric materials is a polyurethane.

In some embodiments, each catheter-tube lumen of the secondary catheter-tube lumen and the tertiary catheter-tube lumen terminates within a medial portion of the first section of the catheter tube.

In some embodiments, an outer layer of the catheter tube is extruded over an inner layer of the catheter tube. The inner layer of the catheter tube includes the reinforcement band thereover.

In some embodiments, the first section of the catheter tube is tapered from the second section of the catheter tube to the catheter tip. The second section of the catheter tube has a constant outer diameter.

Also disclosed herein is a method for making catheter tubes including, in some embodiments, an inner-layer forming step, a banding step, a pulling step, an outer-layer forming step, and a catheter tube-cutting step. The inner-layer forming step includes forming an inner layer of the catheter tube by extruding monoluminal tubing of a first polymeric material with an extruder. The banding step includes periodically banding the monoluminal tubing using a plurality of reinforcement bands to form banded monoluminal tubing. The reinforcement bands are regularly interspersed along the banded monoluminal tubing. The pulling step includes pulling the banded monoluminal tubing through a die of a same or different extruder with a puller. The outer-layer forming step includes periodically forcing a melt of the first polymeric material or a second polymeric material through the die around the banded monoluminal tubing to form output tubing including sections of layered tubing regularly interspersed with sections of the monoluminal tubing. The catheter tube-cutting step includes periodically cutting the output tubing immediately before each section of layered tubing of the sections of layered tubing regularly interspersed with sections of the monoluminal tubing, thereby forming catheter tubes.

In some embodiments, the method further includes a bonding layer-applying step. The bonding layer-applying step includes applying a bonding layer over the monoluminal tubing before forcing the melt of the first or second polymeric material through the die around the banded monoluminal tubing in the outer-layer forming step.

In some embodiments, the method further includes a lumen-forming step. The lumen-forming step includes forming one or more additional lumens to that of the monoluminal tubing by injecting air into the melt of the first or second polymeric material while forcing the melt of the first or second polymeric material through the die around the banded monoluminal tubing in the outer-layer forming step.

In some embodiments, the method further includes an opening-cutting step. The opening-cutting step includes cutting one or more openings in the catheter tubes to correspondingly establish the one-or-more openings for the one-or-more additional lumens.

In some embodiments, the method further includes a tapering step. The tapering step includes periodically increasing a rate of pulling the output tubing with the puller and returning the rate of pulling the output tubing to a constant rate of pulling to respectively form a tapered first section and a constant-diameter second section of each catheter tube of the catheter tubes.

In some embodiments, the first section of each catheter tube of the catheter tubes terminates with a catheter tip reinforced with a reinforcement band of the reinforcement bands.

These and other features of the concepts provided herein will become more apparent to those of skill in the art in view of the accompanying drawings and following description, which describe particular embodiments of such concepts in greater detail.

### DRAWINGS

FIG. 1 illustrates a perspective view of a RICC in accordance with some embodiments.

FIG. 2 illustrates an exploded view of a RICC in accordance with some embodiments.

FIG. 3 illustrates a detailed view of a distal portion a catheter tube of the RICC in accordance with some embodiments.

FIG. 4A illustrates a transverse cross section of the distal portion of the catheter tube of FIG. 3 in accordance with some embodiments.

FIG. 4B illustrates another transverse cross section of the distal portion of the catheter tube of FIG. 3 in accordance with some embodiments.

FIG. 4C illustrates yet another transverse cross section of the distal portion of the catheter tube of FIG. 3 in accordance with some embodiments.

FIG. 4D illustrates yet another transverse cross section of the distal portion of the catheter tube of FIG. 3 in accordance with some embodiments.

FIG. 5A illustrates a longitudinal cross section of the distal portion of the catheter tube of FIG. 3 along a sagittal plane of the RICC in accordance with some embodiments.

FIG. 5B illustrates a longitudinal cross section of the distal portion of the catheter tube of FIG. 3 along a coronal plane of the RICC in accordance with some embodiments.

FIG. 6 illustrates a detailed view of a distal portion another catheter tube of the RICC in accordance with some embodiments.

FIG. 7A illustrates a transverse cross section of the distal portion of the catheter tube of FIG. 6 in accordance with some embodiments.

FIG. 7B illustrates another transverse cross section of the distal portion of the catheter tube of FIG. 6 in accordance with some embodiments.

FIG. 7C illustrates yet another transverse cross section of the distal portion of the catheter tube of FIG. 6 in accordance with some embodiments.

FIG. 7D illustrates yet another transverse cross section of the distal portion of the catheter tube of FIG. 6 in accordance with some embodiments.

FIG. 8 illustrates a method of forming tubing for the catheter tube of FIG. 6 in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or “proximal section” of, for example, a catheter includes a portion or section of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal section, or the proximal length of the catheter can include the proximal end of the catheter; however, the proximal portion, the proximal section, or the proximal length of the catheter need not include the proximal end of the catheter. That is, unless context suggests otherwise, the proximal portion, the proximal section, or the proximal length of the catheter is not a terminal portion or terminal length of the catheter.

With respect to “distal,” a “distal portion” or a “distal section” of, for example, a catheter includes a portion or section of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal section, or the distal length of the catheter can include the distal end of the catheter; however, the distal portion, the distal section, or the distal length of the catheter need not include the distal end of the catheter. That is, unless context suggests otherwise, the distal portion, the distal section, or the distal length of the catheter is not a terminal portion or terminal length of the catheter.

Unless defined otherwise, all technical and scientific terms used herein have the same meaning as commonly understood by those of ordinary skill in the art.

As set forth above, the Seldinger technique is effective for introducing CVCs into patients, but the number of steps are time consuming, handling the number of medical devices is awkward, and both of the foregoing can lead to patient trauma. In addition, there is a relatively high potential for touch contamination due to the number of medical devices that need to be interchanged during the Seldinger technique. As such, there is a need to reduce the number of steps and medical devices involved in introducing a catheter such as a CVC into a patient and advancing the catheter through a vasculature thereof.

Disclosed herein are reinforced catheter tips, catheters including RICCs, and methods thereof that reduce the number of steps and medical devices involved in introducing catheters into patients. In an example, a RICC can include a catheter tube, a catheter hub coupled to a proximal portion of the catheter tube, and a plurality of extension legs extending from the catheter hub. The catheter tube can include a first section in a distal portion of the catheter tube and a second section proximal of the first section. The first section of the catheter tube can distally terminate with a catheter tip reinforced with a reinforcement band therein. The plurality of extension legs that extend from the catheter hub can be equal in number to a plurality of lumens extending through the RICC. In another example, a method can include a method of making the foregoing RICC.

The foregoing features as well as other features of the reinforced catheter tips, catheters (e.g., RICCs), and methods thereof disclosed herein will become more apparent to those of skill in the art in view of the accompanying drawings and following description, which describe particular embodiments of the foregoing in greater detail beginning with RICCs. However, it should be understood CVCs like the RICCs are but one type of catheter that can include the reinforced catheter tips like those disclosed herein. Indeed, other CVCs, peripherally inserted central catheters (“PICCs”), dialysis catheters, or the like can include the reinforced catheter tips for rapidly insertable versions thereof.

### RICCs

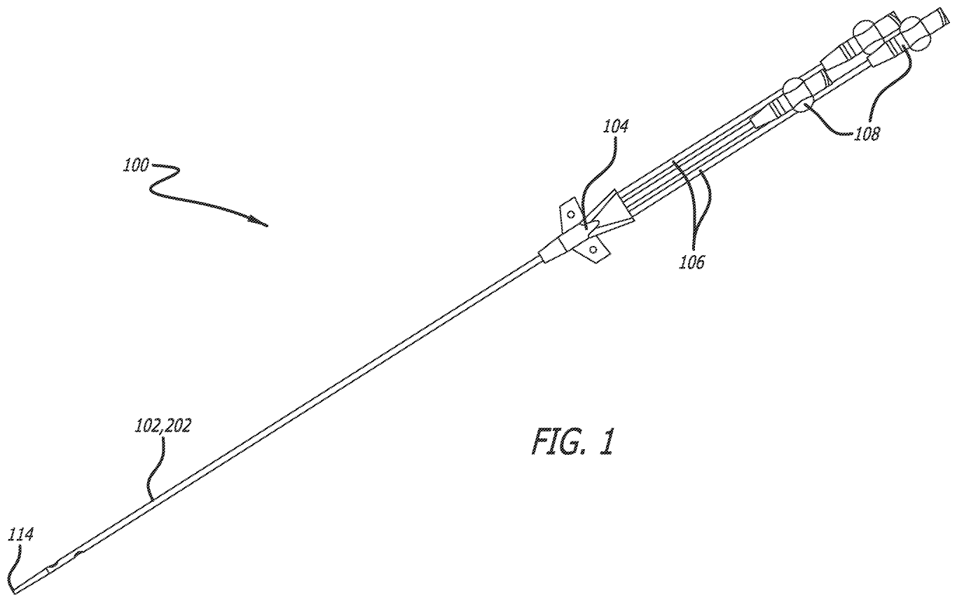

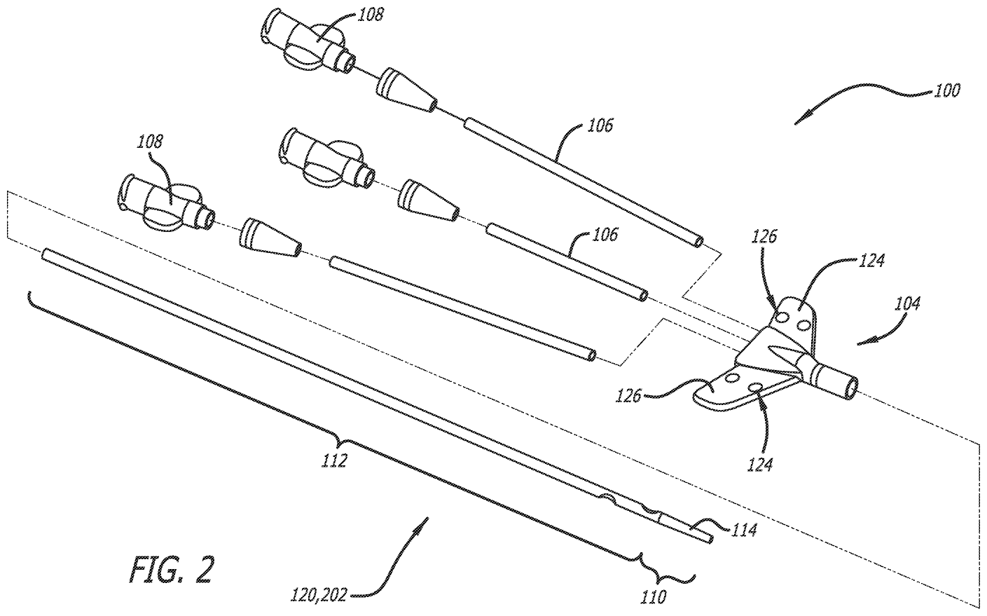

FIGS. 1 and 2 illustrate different views of a RICC 100 in accordance with some embodiments.

As shown, the RICC 100 includes a catheter tube 102 or 202, a catheter hub 104, one or more extension legs 106, and one or more extension-leg connectors 108.

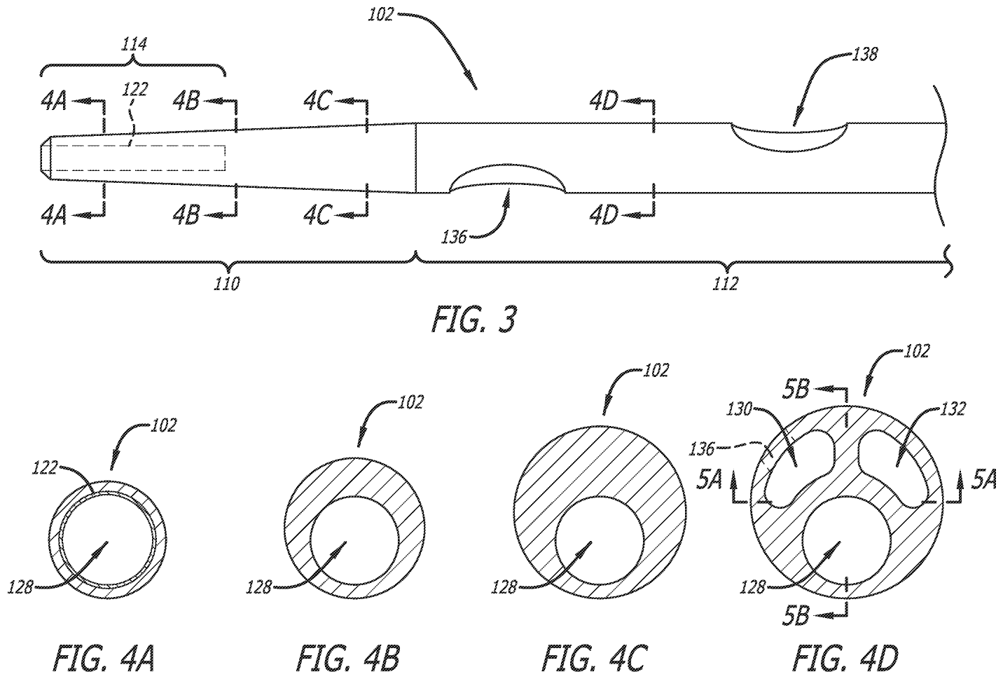

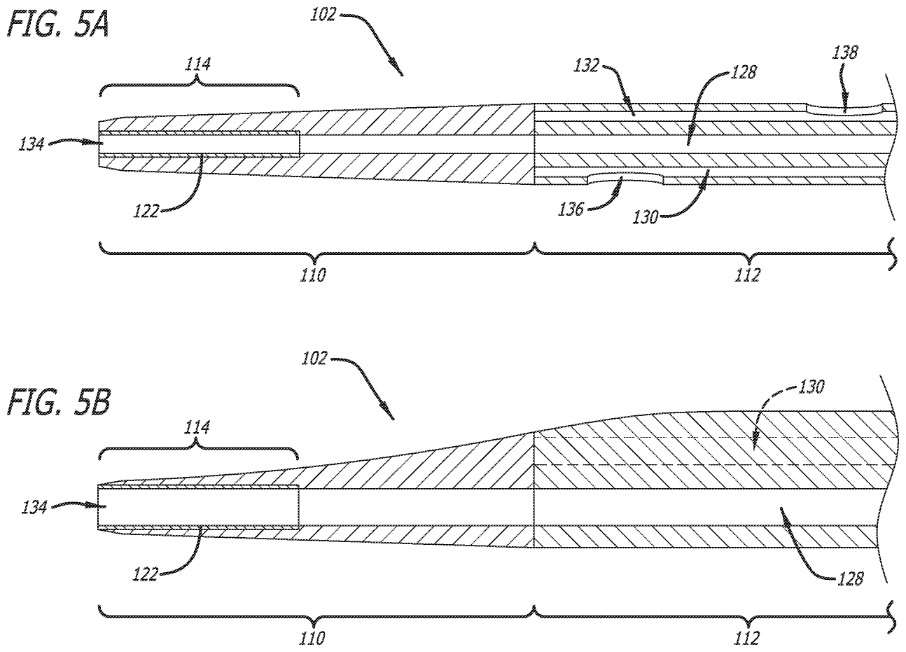

FIGS. 3, 4A-4D, 5A, and 5B illustrate various views of the catheter tube 102 of the RICC 100 in accordance with some embodiments. However, it should be understood CVCs like the RICC 100 are but one type of catheter that can include the catheter tube 102 and the reinforced catheter tip 114 thereof. Indeed, other CVCs, PICCs, dialysis catheters, or the like can include the catheter tube 102 and the reinforced catheter tip 114 thereof for rapidly insertable versions of such catheters.

As shown, the catheter tube 102 includes a first section 110 in a distal portion of the catheter tube 102 and a second section 112 proximal of the first section 110.

The first section 110 of the catheter tube 102 distally terminates with a catheter tip 114 reinforced with a reinforcer therein. In addition, the first section 110 of the catheter tube 102 is tapered from a flat-faced proximal end of the first section 110, which proximal end is fixedly coupled to the distal end of the second section 112 of the catheter tube 102, to a distal end of the first section 110 or the catheter tip 114 thereof. Indeed, the first section 110 of the catheter tube 102 includes a taper from an outer diameter of the proximal end of the first section 110, which outer diameter is commensurate with the outer diameter of the second section 112 of the catheter tube 102, to an outer diameter of the distal end of the first section 110 or the catheter tip 114 thereof. The taper of the first section 110 of the catheter tube 102 is configured for immediate dilation of tissue about a needle tract established with an introducer needle up to the outer diameter of the second section 112 of the catheter tube 102.

The second section 112 of the catheter tube 102 includes a consistent outer diameter over its length from a flat-faced distal end of the second section 112 to a proximal end of the second section 112. The consistent diameter of the second section 112 of the catheter tube 102 is configured for smooth insertion into the foregoing needle tract and targeted vasculature subsequent to any dilation by the first section 110 of the catheter tube 102.

The first and second sections 110 and 112 of the catheter tube 102 are fixedly coupled together at their ends. Indeed, the flat-faced proximal end of the first section 110 of the catheter tube 102 is bonded to the flat-faced distal end of the second section 112 of the catheter tube 102 by a solvent bond, an adhesive bond, or a heat weld. In addition, an abluminal surface of the catheter tube 102 smoothly transitions from an abluminal surface of the first section 110 of the catheter tube 102 to an abluminal surface of the second section 112 of the catheter tube 102 with minimal to negligible edges that do not catch on skin when the catheter tube 102 is inserted into the needle tract. And when solvent bonded, the minimal to negligible edges can include solvent-interdiffused polymeric material of the polymeric materials from which the first and second sections 110 and 112 of the catheter tube 102 are formed, which smoothens the transition from the first section 110 of the catheter tube 102 to the second section 112 of the catheter tube 102.

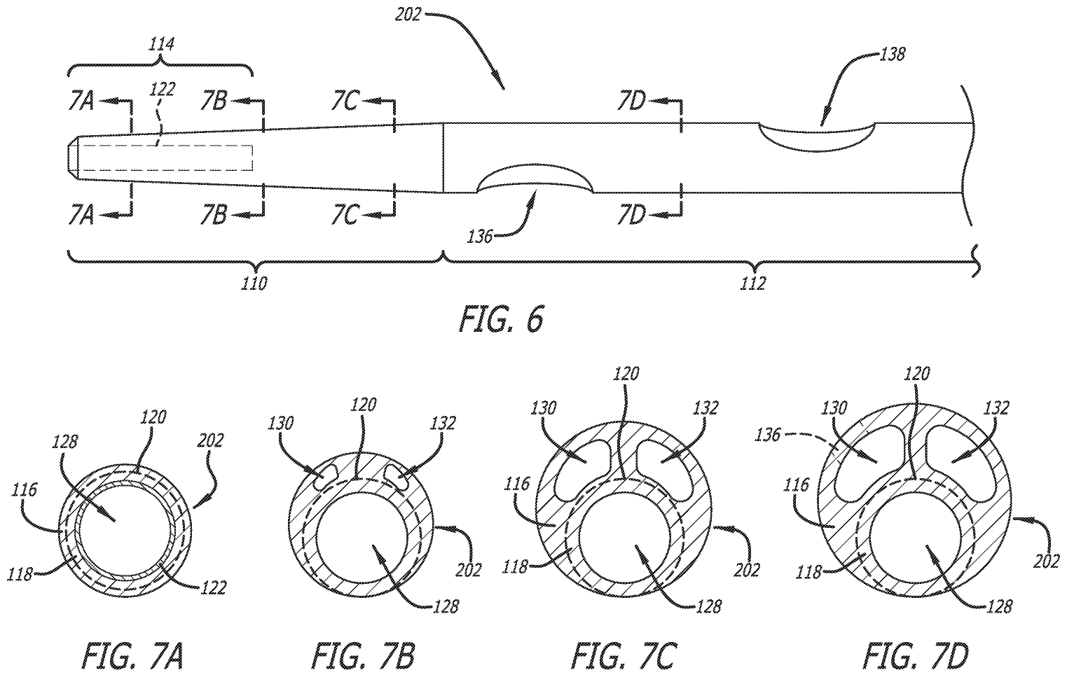

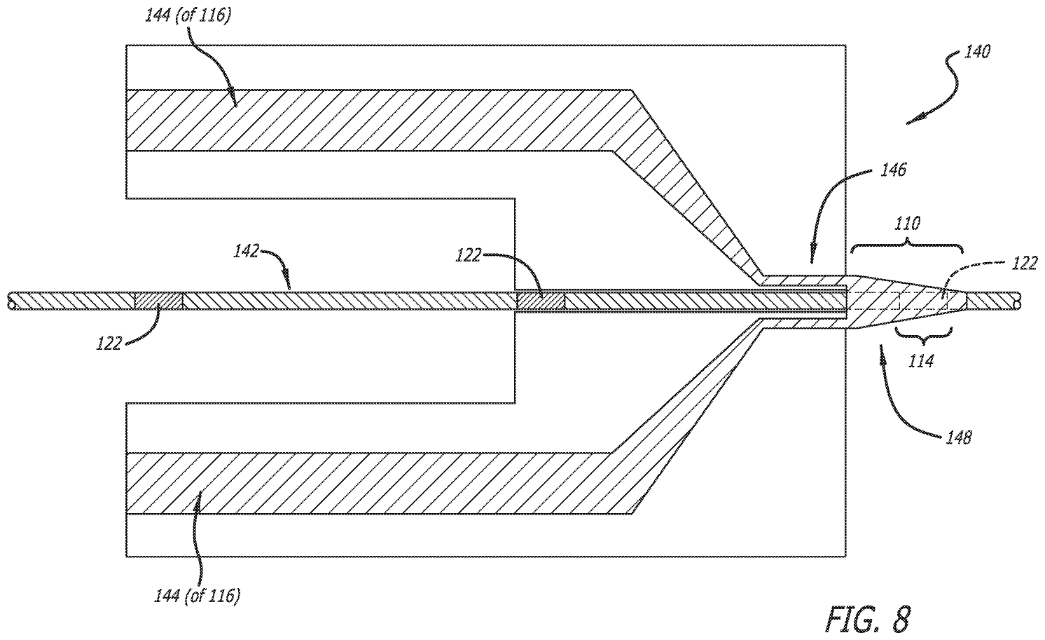

FIGS. 6 and 7A-7D illustrate various views of a catheter tube 202 of the RICC 100 in accordance with some other embodiments. Again, it should be understood CVCs like the RICC 100 are but one type of catheter that can include the catheter tube 202 and the reinforced catheter tip 114 thereof. Indeed, other CVCs, PICCs, dialysis catheters, or the like can include the catheter tube 202 and the reinforced catheter tip 114 thereof for rapidly insertable versions of such catheters.

As shown, the catheter tube 202 is akin to the catheter tube 102 in that it includes the first section 110 in the distal portion of the catheter tube 202 and the second section 112 proximal of the first section 110.

The first section 110 of the catheter tube 202 distally terminates with the catheter tip 114 reinforced with the reinforcer therein. In addition, the first section 110 of the catheter tube 202 is tapered the proximal end of the first section 110 to the distal end of the first section 110 or the catheter tip 114 thereof. Indeed, the first section 110 of the catheter tube 202 includes the taper from the outer diameter of the proximal end of the first section 110, which outer diameter is commensurate with the outer diameter of the second section 112 of the catheter tube 202, to the outer diameter of the distal end of the first section 110 or the catheter tip 114 thereof. Again, the taper of the first section 110 of the catheter tube 202 is configured for immediate dilation of tissue about a needle tract established with an introducer needle up to the outer diameter of the second section 112 of the catheter tube 202.

The second section 112 of the catheter tube 202 includes the consistent outer diameter over its length from distal end of the second section 112 to the proximal end of the second section 112. Again, the consistent diameter of the second section 112 of the catheter tube 202 is configured for smooth insertion into the foregoing needle tract and targeted vasculature subsequent to any dilation by the first section 110 of the catheter tube 202.

Different than the catheter tube 102, the first and second sections 110 and 112 of the catheter tube 202 are not fixedly coupled together at their ends but extruded together in accordance with the method of making the catheter tube 202 set forth below. Indeed, the catheter tube 202 includes an outer layer 116 extruded over an inner layer 118, optionally, with a bonding layer 120 therebetween, wherein the inner layer 118 of the catheter tube 202 is formed from the monoluminal tubing set forth in the method of making the catheter tube 202 below. Notably, on account of extruding the first and second sections 110 and 112 of the catheter tube 202 together, the abluminal surface of the catheter tube 202 smoothly transitions from the abluminal surface of the first section 110 of the catheter tube 202 to the abluminal surface of the second section 112 of the catheter tube 202 with minimal to negligible edges that do not catch on skin when the catheter tube 202 is inserted into the needle tract.

The first section 110 or the inner layer 118 of the catheter tube 102 or 202 can be formed of a first polymeric material (e.g., a polytetrafluoroethylene, a polypropylene, or a polyurethane) having a first durometer. The second section 112 or the outer layer 116 of the catheter tube 102 or 202 can be formed of a second polymeric material (e.g., a polyvinyl chloride, a polyethylene, another polyurethane, or a silicone) having a second durometer less than the first durometer. For example, the first section 110 or the inner layer 118 of the catheter tube 102 or 202 can be formed of a first polyurethane having the first durometer while the second section 112 or the outer layer 116 of the catheter tube 102 or 202 can be formed of a second, different polyurethane (e.g., a same or different diisocyanate or triisocyanate reacted with a different diol or triol, a different diisocyanate or triisocyanate reacted with a same or different diol or triol, a same diisocyanate or triisocyanate reacted with a same diol or triol under different conditions or with different additives, etc.) having the second durometer less than the first durometer. Indeed, polyurethanes are advantageous for the catheter tube 102 or 202 in that polyurethanes can be relatively rigid at room-temperature but become more flexible in vivo at body temperature, which reduces irritation to vessel walls as well as phlebitis. Polyurethanes are also advantageous in that they can be less thrombogenic than some other polymers. Notwithstanding the foregoing, however, the first and second sections 110 and 112 or the inner and outer layers 116 and 118 of the catheter tube 102 or 202 can be formed of a same polymeric material selected from the foregoing polymeric materials.

It should be understood the first durometer of the first polymeric material and the second durometer of the second polymeric material can be on different scales (e.g., Type A or Type D). With this understanding, the second durometer of the second polymeric material might not be numerically less than the first durometer of the first polymeric material when the second durometer is less than the first durometer. Indeed, the hardness of the second polymeric material can still be less than the hardness of the first polymeric material as the different scales—each of which ranges from 0 to 100—are designed for characterizing different materials in groups of the materials having a like hardness.

Adverting back to the catheter tip 114 of the first section 110 of the catheter tube 102 or 202, the catheter tip 114 can include a reinforcement band 122 as the reinforcer within the catheter tip 114. Such a reinforcement band is sufficiently rigid over its length to support aspiration through the catheter tube 102 or 202 or the RICC 100 including the catheter tube 102 or 202 without collapsing the catheter tip 114. Additionally or alternatively, the reinforcement band 122 is sufficiently rigid over its length to prevent buckling of the catheter tip 114 when the catheter tube 102 or 202 or the RICC 100 including the catheter tube 102 or 202 is inserted into the needle tract to a blood-vessel lumen of the target vasculature, optionally, over an access guidewire. The reinforcement band 122 can be formed of a metal or a thermoplastic polymer inserted into a bore of the first section 110 of the catheter tube 102 commensurate with the reinforcement band 122. Alternatively, the first section 110 of the catheter tube 102 can be molded or extruded over the metallic or polymeric reinforcement band 122. Further alternatively, the outer layer 116 of the catheter tube 202 can be extruded over the metallic or polymeric reinforcement band 122 as set forth in the method of making the catheter tube 202 below. When the reinforcement band 122 is formed of the metal, the metal can be selected from at least stainless steel, titanium, and nitinol. When the reinforcement band 122 is formed of the thermoplastic polymer, the thermoplastic polymer can be selected from at least a thermoplastic polyurethane, a carbonate-based thermoplastic polyurethane, a polyamide, and a polyamide-block-polyether copolymer. Advantageously, the thermoplastic polymer of the reinforcement band 122 can be selected to remain relatively rigid in vivo at body temperature in contrast to that set forth above for the first and second sections 110 and 112 or the inner and outer layers 116 and 118 of the catheter tube 102 or 202. In addition, the thermoplastic polymer of the reinforcement band 122 can be formulated to include a radiopacifier such as bismuth trioxide or barium sulfate to enhance radiopacity of the catheter tip 114.

The catheter tube 102 or 202 also includes one or more catheter-tube lumens extending through the catheter tube 102 or 202; however, only one catheter-tube lumen typically extends from a proximal end of the catheter tube 102 or 202 to a distal end of the catheter tube 102 or 202 in a monoluminal or multiluminal RICC (e.g., a diluminal RICC, a triluminal RICC, a tetraluminal RICC, a pentaluminal RICC, a hexaluminal RICC, etc.). Indeed, the first section 110 of the catheter tube 102 or 202 typically includes a single lumen therethrough as shown among FIGS. 3 and 4A-4D for the catheter tube 102 and FIGS. 6 and 7A-7D for the catheter tube 202. Notably, as shown in FIGS. 3 and 4A-4D, each lumen of the secondary lumen 130 and the tertiary lumen 132 terminates at the distal end of the second section 112 of the catheter tube 102. Differently, each lumen of the secondary lumen 130 and the tertiary lumen 132 terminates within a medial portion of the first section 110 of the catheter tube 202 as shown in FIGS. 6 and 7A-7D.

In accordance with the first section 110 or the inner layer 118 of the catheter tube 102 or 202, the second section 112 or the outer layer 116 of the catheter tube 102 or 202, and the catheter tip 114 set forth above, the catheter tube 102 or 202 possesses a column strength, optionally, in combination with an access guidewire disposed therein, sufficient to prevent buckling of the catheter tube 102 or 202 when inserted into a needle tract to a blood-vessel lumen of a patient established by an introducer needle. The column strength of the catheter tube 102 or 202 is also sufficient to prevent buckling of the catheter tube 102 or 202 when advanced through a vasculature of the patient without dilation of tissue about the needle tract or any blood vessels of the vasculature beforehand with a separate dilator. Indeed, the column strength of the catheter tube 102 or 202 is notable in that the column strength makes it possible to rapidly insert the catheter tube 102 or 202 or the RICC 100 including the catheter tube 102 or 202 into a needle tract to a blood-vessel lumen and advance the catheter tube 102 or 202 through the vasculature of the patient without the using the Seldinger technique.

The catheter hub 104 includes a pair of suture wings 124 including a number of suture-wing through holes 126 for suturing the catheter hub 104 to a patient. Each wing of the pair of suture wings 124 can include one, two, three, or four suture-wing through holes 126 for suturing the catheter hub 104 to the patient.

The catheter hub 104 also includes one or more catheter-hub lumens corresponding in number to the one-or-more catheter-tube lumens. The one-or-more catheter-hub lumens extends through an entirety of the catheter hub 104 from a proximal end of the catheter hub 104 to a distal end of the catheter hub 104. When the catheter hub 104 is coupled to a proximal portion of the catheter tube 102 or 202 as shown in FIG. 1, the one-or-more catheter-tube lumens continue through the one-or-more catheter-hub lumens, respectively.

Each extension leg of the one-or-more extension legs 106 is coupled to—and, therefore, extends from—the catheter hub 104 by a distal portion thereof. The one-or-more extension legs 106 respectively include one or more extension-leg lumens, which, in turn, correspond in number to the one-or-more catheter-hub lumens. Each extension-leg lumen of the one-or-more extension-leg lumens extends through an entirety of the extension leg from a proximal end of the extension leg to a distal end of the extension leg. When the one-or-more extension legs 106 are coupled to the catheter hub 104 as shown in FIG. 1, the one-or-more catheter-hub lumens continue through the one-or-more extension-leg lumens, respectively.

Each extension-leg connector of the one-or-more extension-leg connectors 108 is over a proximal portion of an extension leg of the one-or-more extension legs 106. For example, each extension-leg connector of the one-or-more extension-leg connectors 108 can be a Luer connector over a proximal portion of an extension leg of the one-or-more extension legs 106. Through such an extension-leg connector, a corresponding extension leg and the extension-leg lumen thereof can be connected to another medical device and a lumen thereof.

The RICC 100 can be a monoluminal RICC or a multiluminal RICC such as a diluminal RICC, a triluminal RICC, a tetraluminal RICC, a pentaluminal RICC, a hexaluminal RICC, etc. When the RICC 100 is configured as the triluminal RICC shown in FIGS. 1 and 2, the RICC 100 includes a set of three lumens. The set of three lumens includes a primary lumen 128, a secondary lumen 130, and a tertiary lumen 132 formed of fluidly connected portions of three catheter-tube lumens of the catheter tube 102 or 202, three catheter-hub lumens of a trifurcated hub for the catheter hub 104, and three extension-leg lumens of three extension legs 106. The primary lumen 128 has a primary-lumen opening 134 in the distal end of the first section 110 of the catheter tube 102 or 202 or the catheter tip 114 thereof, which corresponds to a distal end of the RICC 100. The secondary lumen 130 has a secondary-lumen opening 136 in a side of the distal portion of the catheter tube 102 or 202. The tertiary lumen 132 has a tertiary-lumen opening 138 in the side of the distal portion of the catheter tube 102 or 202 proximal of the secondary-lumen opening 136.

### Methods

Methods include a method for making the catheter tube 102 or 202 of the RICC 100 set forth above. For example, a method of making the catheter tube 202 includes one or more steps selected from an inner-layer forming step, a banding step, a bonding layer-applying step, a pulling step, an outer-layer forming step, a tapering step, a lumen-forming step, a catheter tube-cutting step, and an opening-cutting step.

The inner-layer forming step includes forming the inner layer 118 of the catheter tube 202 by extruding monoluminal tubing of the first polymeric material with an extruder 140.

The banding step includes periodically banding the monoluminal tubing using a plurality of reinforcement bands to form banded monoluminal tubing 142. The reinforcement bands are regularly interspersed along the banded monoluminal tubing 142.

The bonding layer-applying step includes applying the bonding layer 120 over the monoluminal tubing or the banded monoluminal tubing 142 before forcing a melt 144 of the first or second polymeric material through a die 146 of the extruder 140 around the banded monoluminal tubing 142 in the outer-layer forming step.

The pulling step includes pulling the banded monoluminal tubing 142 through the die 146 of a same or different extruder with a puller (not shown).

The outer-layer forming step includes periodically forcing the melt 144 of the first polymeric material or the second polymeric material through the die 146 around the banded monoluminal tubing 142 to form output tubing 148 including sections of layered tubing regularly interspersed with sections of the monoluminal tubing.

The tapering step includes periodically increasing a rate of pulling the output tubing 148 with the puller and returning the rate of pulling the output tubing 148 to a constant rate of pulling to respectively form the tapered first section 110 and the constant-diameter second section 112 of each catheter tube of the catheter tubes.

The lumen-forming step includes forming one or more additional lumens to that of the monoluminal tubing by injecting air into the melt 144 of the first or second polymeric material while forcing the melt 144 of the first or second polymeric material through the die 146 around the banded monoluminal tubing 142 in the outer-layer forming step.

The catheter tube-cutting step includes periodically cutting the output tubing 148 immediately before each section of layered tubing of the sections of layered tubing regularly interspersed with sections of the monoluminal tubing, thereby forming the catheter tubes. The first section 110 of each catheter tube of the catheter tubes terminates with the catheter tip 114 reinforced with the reinforcement band 122 of the reinforcement bands.

The opening-cutting step includes cutting (e.g., laser cutting) one or more openings in the catheter tubes to correspondingly establish the one-or-more openings (e.g., the secondary-lumen opening 136, the tertiary-lumen opening 138, etc.) for the one-or-more additional lumens.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

1. A rapidly insertable central catheter (“RICC”), comprising:
a catheter tube, including:
a first section in a distal portion of the catheter tube, the first section of the catheter tube distally terminating with a catheter tip reinforced with a reinforcement band therein; and
a second section proximal of the first section of the catheter tube;
a catheter hub coupled to a proximal portion of the catheter tube; and
a plurality of extension legs extending from the catheter hub equal in number to a plurality of lumens extending through the RICC.

2. The RICC of claim 1, wherein the reinforcement band is sufficiently rigid over its length to support aspiration through the RICC without collapsing the catheter tip.

3. The RICC of claim 1, wherein the reinforcement band is sufficiently rigid over its length to prevent buckling of the catheter tip when the RICC is inserted into a needle tract to a blood-vessel lumen, optionally, over an access guidewire.

4. The RICC of claim 1, wherein the reinforcement band is formed of a thermoplastic polymer or a metal over which the first section of the catheter tube is molded or extruded.

5. The RICC of claim 4, wherein the reinforcement band is formed of the thermoplastic polymer, the thermoplastic polymer selected from a thermoplastic polyurethane, a carbonate-based thermoplastic polyurethane, a polyamide, and a polyamide-block-polyether copolymer.

6. The RICC of claim 4, wherein the reinforcement band is formed of the metal, the metal selected from stainless steel, titanium, and nitinol.

7. The RICC of claim 1, wherein the RICC is a triluminal catheter including a trifurcated catheter hub as the catheter hub and three extension legs for the plurality of extension legs, each extension leg of the three extension legs including a Luer connector coupled to a proximal portion of the extension leg.

8. The RICC of claim 7, wherein the RICC includes a primary lumen extending from an opening in a proximal end of a first Luer connector to an opening in the catheter tip, a secondary lumen extending from an opening in a proximal end of a second Luer connector to a secondary-lumen opening in a distal portion of the second section of the catheter tube, and a tertiary lumen extending from an opening in a proximal end of a third Luer connector to a tertiary-lumen opening in the distal portion of the second section of the catheter tube proximal of the secondary-lumen opening.

9. The RICC of claim 8, wherein each lumen of the secondary lumen and the tertiary lumen terminates at a distal end of the second section of the catheter tube.

10. The RICC of claim 9, wherein a proximal end of the first section of the catheter tube is bonded to the distal end of the second section of the catheter tube.

11. The RICC of claim 8, wherein each lumen of the secondary lumen and the tertiary lumen terminates within a medial portion of the first section of the catheter tube.

12. The RICC of claim 11, wherein an outer layer of the catheter tube is extruded over an inner layer of the catheter tube, the inner layer of the catheter tube including the reinforcement band thereover.

13. The RICC of claim 1, wherein the first section of the catheter tube is formed of a first polymeric material having a first durometer and the second section of the catheter tube is formed of a second polymeric material having a second durometer less than the first durometer.

14. The RICC of claim 13, wherein each polymeric material of the first and second polymeric materials is a polyurethane.

15. The RICC of claim 1, wherein the first section of the catheter tube is tapered from the second section of the catheter tube to the catheter tip, the second section of the catheter tube having a constant outer diameter.

16. A catheter tube, comprising:
a first section in a distal portion of the catheter tube having a single catheter-tube lumen passing therethrough, the first section of the catheter tube distally terminating with a catheter tip reinforced with a reinforcement band therein; and
a second section proximal of the first section of the catheter tube, the second section of the catheter tube having two or more catheter-tube lumens passing therethrough including a same catheter-tube lumen as the catheter-tube lumen passing through the first section of the catheter tube.

17. The catheter tube of claim 16, wherein the reinforcement band is sufficiently rigid over its length to support aspiration through the catheter tube without collapsing the catheter tip.

18. The catheter tube of claim 16, wherein the reinforcement band is sufficiently rigid over its length to prevent buckling of the catheter tip when the catheter tube is inserted into a needle tract to a blood-vessel lumen, optionally, over an access guidewire.

19. The catheter tube of claim 16, wherein the reinforcement band is formed of a thermoplastic polymer or a metal over which the first section of the catheter tube is molded or extruded.

20. The catheter tube of claim 19, wherein the reinforcement band is formed of the thermoplastic polymer, the thermoplastic polymer selected from a thermoplastic polyurethane, a carbonate-based thermoplastic polyurethane, a polyamide, and a polyamide-block-polyether copolymer.

21. The catheter tube of claim 19, wherein the reinforcement band is formed of the metal, the metal selected from stainless steel, titanium, and nitinol.

22. The catheter tube of claim 16, wherein the catheter tube is a triluminal catheter tube including:
a primary catheter-tube lumen for the catheter-tube lumen passing through both the first and second sections of the catheter tube, the primary catheter-tube lumen extending from an opening in a proximal end of the second section of the catheter tube to an opening in the catheter tip;
a secondary catheter-tube lumen extending from an opening in the proximal end of the second section of the catheter tube to a secondary-lumen opening in a distal portion of the second section of the catheter tube; and
a tertiary catheter-tube lumen extending from an opening in the proximal end of the second section of the catheter tube to a tertiary-lumen opening in the distal portion of the second section of the catheter tube proximal of the secondary-lumen opening.

23. The catheter tube of claim 22, wherein each catheter-tube lumen of the secondary catheter-tube lumen and the tertiary catheter-tube lumen terminates at a distal end of the second section of the catheter tube.

24. The catheter tube of claim 23, wherein a proximal end of the first section of the catheter tube is bonded to the distal end of the second section of the catheter tube.

25. The catheter tube of claim 22, wherein each catheter-tube lumen of the secondary catheter-tube lumen and the tertiary catheter-tube lumen terminates within a medial portion of the first section of the catheter tube.

26. The catheter tube of claim 25, wherein an outer layer of the catheter tube is extruded over an inner layer of the catheter tube, the inner layer of the catheter tube including the reinforcement band thereover.

27. The catheter tube of claim 16, wherein the first section of the catheter tube is formed of a first polymeric material having a first durometer and the second section of the catheter tube is formed of a second polymeric material having a second durometer less than the first durometer.

28. The catheter tube of claim 27, wherein each polymeric material of the first and second polymeric materials is a polyurethane.

29. The catheter tube of claim 16, wherein the first section of the catheter tube is tapered from the second section of the catheter tube to the catheter tip, the second section of the catheter tube having a constant outer diameter.

30-35. (canceled)
